(19) United States
(12) Patent Application Publication
(10) Pub. No.: US 2005/0004039 A1
(43) Pub. Date: Jan. 6, 2005

(54) Selective analgesic agents

(75) Inventor: Philip S. Portoghese, St. Paul, MN (US)

Correspondence Address: Schwegman, Lundberg, Woessner & Kluth, P.A., P.O. Box 2938, Minneapolis, MN, 55402, US

(73) Assignee: Regents of the University of Minnesota

(21) Appl. No.: 10/898,802

(22) Filed: Jul. 26, 2004

Related U.S. Application Data
(63) Continuation of application No. PCT/US03/02257, filed on Jan. 27, 2003.
(60) Provisional application No. 60/352,030, filed on Jan. 25, 2002.

(51) Int. Cl. A61K 38/10; A61K 31/485

(52) U.S. Cl. 514/17; 514/282

(57) ABSTRACT

The invention provides a method for producing a selective analgesic effect outside the brain (e.g. via opioid receptors in the spinal cord) of a mammal, comprising administering to the mammal, an effective analgesic dose of a compound that binds to delta-kappa opioid receptors in the mammal.

### GOVERNMENT FUNDING

The invention described herein was made with U.S. Government support under Grant Number DA01533 awarded by the National Institute on Drug Abuse. The United States Government has certain rights in the invention.

### BACKGROUND OF THE INVENTION

Endogenous opioid peptides are known and are involved in the mediation or modulation of a variety of mammalian physiological processes, many of which are mimicked by opiates or other non-endogenous opioid ligands. Some of the effects that have been suggested include analgesia, tolerance and dependence, appetite, renal function, gastrointestinal motility, gastric secretion, learning and memory, mental illness, epileptic seizures and other neurological disorders, cardiovascular responses, and respiratory depression.

Heterodimers of kappa and delta opioid receptors have been reported to possess ligand binding properties that differ from those of either receptor (see S. George et al., J. Biol. Chem., 2000, 275, 26128-26135; and B. A. Jordan et al., Nature, 1999, 399, 697-700). In this regard, the possibility has been raised that some opioid receptor subtypes may be heterodimers (see B. A. Jordan et al., Neuropsychopharmacol, 2000, 23, S5-S18).

Additionally, kappa and delta receptors have been reported to be coexpressed in single axons in the spinal cord (see M. W. Wessendorf et al., Neurosci. Lett., 2001, 298, 151-154). Taken together with the report that intrathecal co-injection of selective kappa and delta agonists produced antinociceptive synergy in rats (see C. Miaskowski et al., Brain Res., 1990, 509, 165-168), the data are consistent with the existence of heterodimers or hetero-oligomers in vivo. Also, similar co-localization of kappa and delta receptors in the porcine ileum has been reported (see S. Poonyachoti et al., J. Pharmacol exp. Ther., 2001, 297, 69-77.

There exists a need for a method for producing selective analgesic effects outside the brain (e.g. spinal analgesia) of a mammal without producing analgesic effects in the brain, as many of the side-effects associated with opioid analgesics are mediated via receptors in the brain.

### SUMMARY OF THE INVENTION

Applicant has discovered that it is possible to selectively target delta-kappa opioid receptors in the spinal cord (and other tissues) with appropriate ligands to selectively produce analgesia. Such ligands do not produce significant brain mediated analgesia. In particular, the ligand, 6′-guanidino-naltrindole (6′-GNTI, compound 7) is selective for the putative kappa-delta opioid receptor dimer in the spinal cord.

Accordingly, the invention provides a method for producing a selective analgesic effect outside the brain in a mammal, comprising administering to the mammal an effective analgesic dose of a compound that activates a delta-kappa opioid receptor in the mammal.

The invention also provides a method for producing an analgesic effect in a mammal via selective agonism of opioid receptors in the spinal cord of the mammal, comprising administering to the mammal, an effective analgesic dose of a compound that selectively activates delta-kappa opioid receptors.

The invention also provides a method for producing spinal analgesia in a mammal comprising, administering to the mammal, an effective analgesic dose of a compound that activates delta-kappa opioid receptors.

The invention also provides a method to produce selective agonism of opioid receptors outside the brain in a mammal comprising administering to the mammal an effective dose of a compound that activates delta-kappa opioid receptors.

The invention also provides a method to produce spinal analgesia in a mammal comprising administering to the mammal an effective dose of a compound that selectively activates opioid receptors in the spine.

The invention also provides novel compounds of formulas (I) disclosed herein (e.g. a compound of formula (I) wherein X is CH₂ as well as a compound of formula (I) wherein R has any of the values, specific values, or preferred values described herein other than hydrogen), as well as intermediates and processes described herein which are useful for preparing compounds of formula (I) or (II).

The invention also provides a pharmaceutical composition comprising the novel compounds of the invention or compounds useful in the methods of the invention and a pharmaceutical carrier.

The invention also provides the use of a compound that selectively activates a delta-kappa opioid receptor for the manufacture of a medicament useful for producing a selective analgesic effect outside the brain in a mammal.

The invention also provides the invention is the use of a compound that selectively activates delta-kappa opioid receptors for the manufacture of a medicament useful for producing an analgesic effect in a mammal via selective agonism of opioid receptors in the spinal cord of a mammal.

The invention also provides the use of a compound that activates delta-kappa opioid receptors for the manufacture of a medicament for producing spinal analgesia in a mammal.

The invention also provides the use of a compound that activates delta-kappa opioid receptors for the manufacture of a medicament for producing selective agonism of opioid receptors outside the brain in a mammal.

The invention also provides the use of a compound that selectively activates opioid receptors in the spine for the manufacture of a medicament for producing spinal analgesia in a mammal.

The invention also provides the use of a compound of the invention in a mammal, wherein the mammal is a human.

The invention also provides a method for identifying an analgesic agent capable of producing a selective analgesic effect outside the brain in a mammal, comprising determining if the compound activates a delta-kappa opioid receptor.

The invention also provides a method for identifying a compound capable of producing an analgesic effect in a mammal via selective agonism of opioid receptors in the spinal cord of a mammal comprising determining if the compound selectively activates delta-kappa opioid receptors over kappa-, mu- or delta- opioid receptors.

The invention also provides a method for identifying a compound capable of producing spinal analgesia in a mammal, comprising determining if the compound activates a delta-kappa opioid receptor.

The invention also provides a method for identifying a compound capable of selective agonism of opioid receptors outside the brain in a mammal comprising determining if the compound has a greater agonist effect on opioid receptors outside the brain than its agonist effect on opioid receptors inside the brain.

The invention also provides a method for identifying a compound capable of producing spinal analgesia in a mammal, comprising determining if the compound selectively activates opioid receptors in the spine of the mammal.

### BRIEF DESCRIPTION OF THE FIGURES

FIG. 1 Illustrates the synthesis of representative compounds of formula (I).

FIG. 2 Illustrates general synthetic methods useful for preparing compounds of formula (I).

FIG. 3 Illustrates the structure of compound 7 (6′-GNTI).

FIG. 4 Shows the concentration-response curve of compound 7 compared to that of morphine in a guinea-pig ileum preparation.

FIG. 5 Illustrates the preparation of representative compounds of formula (I) (e.g. compounds 1, 2a, 2b, 3a-3d, 4a-4d, 5, 6a-6b, and 7).

FIG. 6 Illustrates the preparation of representative compounds of formula (1) (e.g. compounds 21a, 21b, 22a, 22b, 23a and 23b).

FIG. 7 Shows the antinociception induced by DAMGO in the presence or absence of nor-BNI and with or without antiserum to nor-BNI.

FIG. 8 Shows the antinociception induced by DPDPE in the presence or absence of nor-BNI and with or without antiserum to nor-BNI.

### DETAILED DESCRIPTION

Definitions

A “delta-kappa opioid receptor,” as used herein, refers to a receptor complex that comprises at least one delta subunit and at least one kappa subunit. In a preferred embodiment, a delta-kappa opioid receptor contains only delta and kappa subunits. In other embodiments, it can contain other opioid receptor subunits, such as mu receptor subunits.

A review referencing papers reporting the cloning and sequencing of cDNA and genomic clones of human and other mammalian delta and kappa receptor polypeptides is Dhawan et al., Pharmacol. Rev. 48:567-692 (1996). For instance, the nucleotide sequence of delta mRNA is provided in GenBank accession number U07882, and the amino acid sequence of the kappa protein in accession number AAA18789. The nucleotide sequence of the kappa mRNA is found in GenBank accession number U17298, and the protein amino acid sequence in accession number JC2338. A mammalian delta opioid receptor polypeptide can be identified by its binding to and agonism by known delta agonists. A mammalian kappa opioid receptor polypeptide can be identified by its binding to and agonism by known kappa agonists. Mammalian delta opioid receptors typically have greater than about 90% amino acid sequence identity with human delta opioid receptor. Mammalian kappa opioid receptors typically have greater than about 90% amino acid sequence identity with human kappa opioid receptor. Amino acid sequence identity can be calculated with BLAST 2.0 using the default parameters, as available at www.ncbi.nlm.nih.gov.

“Activation of a delta-kappa opioid receptor,” as used herein, refers to an induction of a biological effect through the binding of an agent to one or more of the subunits of a delta-kappa opioid receptor. The biological effect could be a behavioral or sensory effect, such as a reduction in the sensation of pain or induction of euphoria or an increased sense of well-being. The biological effect can also be a physiological effect, such as a reduction in the firing of neurons, or a biochemical effect, such as an alteration in membrane polarization, glutamate release, or intracellular calcium release, or an activation of adenyl cyclase.

As used herein, the term “selective agonism of opioid receptor in the spinal cord” refers to the greater agonism of opioid receptors in the spinal cord than opioid receptors in one or more other parts of the neurological system, such as the brain. This can occur, for instance, by selective agonism of a receptor type that is present in greater amounts in the spinal cord than in other parts of the neurological system.

Description

In particular embodiments of the invention involving administering to a mammal a compound that activates opioid receptors or delta-kappa opioid receptors, the compound binds to a delta-kappa opioid receptor at least 3-fold more strongly than it binds to a kappa receptor. In other specific embodiments, the compound binds to a delta-kappa opioid receptor at least 5-fold or at least 10-fold more strongly than it binds to a kappa receptor.

In a specific embodiment of the methods of the invention involving administering to a mammal a compound that activates opioid receptors or delta-kappa opioid receptors, the compound binds to a delta-kappa opioid receptor at least 3-fold, at least 5-fold, or at least 10-fold more strongly than it binds to a delta receptor.

In a specific embodiment of the methods of the invention involving administering to a mammal a compound that activates opioid receptors or delta-kappa opioid receptors, the compound binds to a delta-kappa opioid receptor at least 3-fold, at least 5-fold, or at least 10-fold more strongly than it binds to a mu receptor.

In a specific embodiment of the method of the invention involving administering to a mammal a compound that activates opioid receptors or delta-kappa opioid receptors, the compound is administered orally. In another specific embodiment, the compound is administered intrathecally. In another specific embodiment, the compound is not administered intrathecally.

In a specific embodiment of the invention, the compound administered is [D-Pen^{2,5}]enkephalin (DPDPE). In another specific embodiment, the compound is not DPDPE. In a specific embodiment, the compound is not a peptide.

In one embodiment, a compound that can be administered according to the methods of the invention is a compound of formula (I): 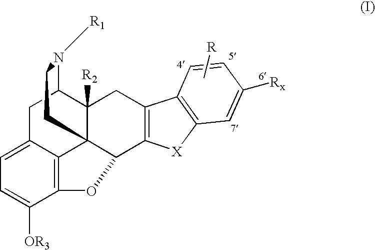
wherein:
R is hydrogen, halo, hydroxy, nitro, cyano, trifluoromethyl, trifluoromethoxy, (C₁-C₆)alkyl, (C₂-C₆)alkenyl, (C₂-C₆)alkynyl, (C₃-C₇)cycloalkyl, (C₅-C₇)cycloalkenyl, (C₃-C₆)cycloalkyl(C₁-C₆)alkyl, (C₅-C₇)cycloalkenyl (C₁-C₆)alkyl, aryl, heteroaryl, aryl(C₁-C₆)alkyl, heteroaryl(C₁-C₆)alkyl (C₁-C₆)alkoxy, (C₁-C₆)alkanoyloxy, NRₐR_{b} or SR_{c};
R₁ is (C₁-C₆)alkyl, (C₂-C₆)alkenyl, (C₂-C₆)alkynyl, (C₃-C₇)cycloalkyl, (C₅-C₇)cycloalkenyl, (C₃-C₆)cycloalkyl(C₁-C₆)alkyl, (C₅-C₇)cycloalkenyl (C₁-C₆)alkyl, aryl, heteroaryl, aryl(C₁-C₆)alkyl, or heteroaryl(C₁-C₆)alkyl;
R₂ is H, hydroxy, (C₁-C₆)alkoxy, (C₁-C₆)alkanoyloxy, NRₐR_{b} or SR_{c};
R₃ is H, aryl(C₁-C₆)alkyl, (C₁-C₆)alkyl, (C₁-C₆)alkanoyl, or (C₁-C₆)alkylC(═S);
Rₓ is a basic or positively charged group (i.e. a group that is positively charged or that is capable of being positively charged under physiological conditions) or an organic radical that comprises a basic or positively charged group;
X is O, S, CH₂, or NY;
Y is H, (C₁-C₆)alkyl, or aryl(C₁-C₆)alkyl; and
Rₐ—R_{c} are each independently H, (C₁-C₆)alkyl, (C₁-C₆)alkanoyl, phenyl, benzyl, phenethyl, or —C(═S)(C₁-C₆)alkyl;
or a pharmaceutically acceptable salt thereof.

In a particular embodiment, the basic or positively charged group of the compound of formula (I) is a quaternary amine or an amine salt.

Another compound that binds to delta-kappa opioid receptors, which can be administered according to the methods of the invention is a compound of formula (II): 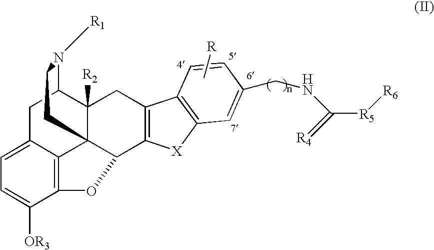
wherein:
R is hydrogen, halo, hydroxy, nitro, cyano, trifluoromethyl, trifluoromethoxy, (C₁-C₆)alkyl, (C₂-C₆)alkenyl, (C₂-C₆)alkynyl, (C₃-C₇)cycloalkyl, (C₅-C₇)cycloalkenyl, (C₃C₆)cycloalky(C₁-C₆)alkyl, (C₅-C₇)cycloalkenyl(C₁-C₆)alkyl, aryl, heteroaryl, aryl(C₁-C₆)alkyl, heteroaryl(C₁-C₆)alkyl (C₁-C₆)alkoxy, (C₁-C₆)alkanoyloxy, NRₐR_{b} or SR_{c};
R₁ is (C₁-C₆)alkyl, (C₂-C₆)alkenyl, (C₂-C₆)alkynyl, (C₃-C₇)cycloalkyl, (C₅-C₇)cycloalkenyl, (C₃-C₆)cycloalkyl(C₁-C₆)alkyl, (C₅-C₇)cycloalkenyl(C₁-C₆)alkyl, aryl, heteroaryl, aryl(C₁-C₆)alkyl, or heteroaryl(C₁-C₆)alkyl;
R₂ is H, OH, (C₁-C₆)alkoxy, (C₁-C₆)alkanoyloxy, NRₐR_{b} or SR_{c};
R₃ is H, aryl(C₁-C₆)alkyl, (C₁-C₆)alkyl, (C₁-C₆)alkanoyl, or (C₁-C₆)alkylC(═S);
R₄ is ═O, ═S, or ═NR_{d};
R_{d} is H, CN, CONH₂, COCF₃, (C₁-C₆)alkanoyl, (C₁-C₆)alkyl, or (CH₂)ₚNRₑR_{f}, or R_{d} together with R₆ is —(CH₂)_{q}— and forms a ring;
p is 1, 2, 3, or 4;
R₅ is NRₘ;
R₆ is H, (C₁-C₆)alkyl, (C₃-C₇)cycloalkyl, aryl, heteroaryl, aryl(C₁-C₆)alkyl, heteroaryl(C₁-C₆)alkyl, NR_{g}Rₕ(C₁-C₆)alkyl, or C(═NRⱼ)NHRₖ; or when R₄ is ═NR_{d}, R₆ together with R_{d} is —(CH₂)_{q}— and forms a ring;
q is 2 or 3;
X is O, S, CH₂, or NY;
Y is H, (C₁-C₆)alkyl, or aryl(C₁-C₆)alkyl;
n is O, 1, 2, 3, or 4;
Rₐ—R_{c} and Rₑ—R_{f} are each independently H, (C₁-C₆)alkyl, (C₁-C₆)alkanoyl, phenyl, benzyl, phenethyl, or —C(═S)(C₁-C₆)alkyl;
R_{g} and Rₕ are each independently H, (C₁-C₆)alkyl, (C₁-C₆)alkanoyl, —C(═NH)NRₐR_{b}, or —C(═S)(C₁-C₆)alkyl, or R_{g} and Rₕ together with the nitrogen to which they are attached are pyrrolidino, piperidino or morpholino;
Rⱼ and Rₖ are each independently H, (C₁-C₆)alkyl, (C₂-C₆)alkenyl, (C₂-C₆)alkynyl, (C₃-C₇)Cycloalkyl, (C₃-C₇)cycloalkyl(C₁-C₆)alkyl, (C₅-C₇)cycloalkenylalkyl, aryl, heteroaryl, aryl(C₁-C₆)alkyl, or heteroaryl(C₁-C₆)alkyl; and
Rₘ is hydrogen or (C₁-C₆)alkyl;
or a pharmaceutically acceptable salt thereof.

A particular compound that can be administered according to the invention is a compound of formula (I) wherein: R is hydrogen, halo, hydroxy, nitro, cyano, trifluoromethyl, trifluoromethoxy, (C₁-C₆)alkyl, (C₂-C₆)alkenyl, (C₂-C₆)alkynyl, (C₃-C₇)cycloalkyl, (C₅-C₇)cycloalkenyl, (C₃-C₆)cycloalkyl(C₁-C₆)alkyl, (C₅-C₇)cycloalkenyl(C₁-C₆)alkyl, aryl, heteroaryl, aryl(C₁-C₆)alkyl, heteroaryl(C₁-C₆)alkyl (C₁-C₆)alkoxy, (C₁-C₆)alkanoyloxy, NRₐR_{b} or SR_{c}; R₁ is (C₁-C₆)alkyl, (C₂-C₆)alkenyl, (C₂-C₆)alkynyl, (C₃-C₇)cycloalkyl, (C₅-C₇)cycloalkenyl, (C₃-C₆)cycloalkyl(C₁-C₆)alkyl, (C₅-C₇)cycloalkenyl(C₁-C₆)alkyl, aryl, heteroaryl, aryl(C₁-C₆)alkyl, or heteroaryl(C₁-C₆)alkyl; R₂ is H, hydroxy, (C₁-C₆)alkoxy, (C₁-C₆)alkanoyloxy, NRₐR_{b} or SR_{c}; R₃ is H, aryl(C₁-C₆)alkyl, (C₁-C₆)alkyl, (C₁-C₆)alkanoyl, or (C₁-C₆)alkylC(═S); Rₓ is a basic or positively charged group or an organic radical that comprises a basic or positively charged group; X is CH₂; and Rₐ—R_{c}, are each independently H, (C₁-C₆)alkyl, (C₁-C₆)alkanoyl, phenyl, benzyl, phenethyl, or —C(═S)(C₁-C₆)alkyl; or a pharmaceutically acceptable salt thereof.

The invention also provides a novel compound of formula (I) wherein: R is halo, hydroxy, nitro, cyano, trifluoromethyl, trifluoromethoxy, (C₁-C₆)alkyl, (C₂-C₆)alkenyl, (C₂-C₆)alkynyl, (C₃-C₇)cycloalkyl, (C₅-C₇)cycloalkenyl, (C₃-C₆)cycloalkyl(C₁-C₆)alkyl, (C₅-C₇)cycloalkenyl(C₁-C₆)alkyl, aryl, heteroaryl, aryl(C₁-C₆)alkyl, heteroaryl(C₁-C₆)alkyl (C₁-C₆)alkoxy, (C₁-C₆)alkanoyloxy, NRₐR_{b} or SR_{c}; R₁ is (C₁-C₆)alkyl, (C₂-C₆)alkenyl, (C₂-C₆)alkynyl, (C₃-C₇)cycloalkyl, (C₅-C₇)cycloalkenyl, (C₃-C₆)cycloalkyl(C₁-C₆)alkyl, (C₅-C₇)cycloalkenyl(C₁-C₆)alkyl, aryl, heteroaryl, aryl(C₁-C₆)alkyl, or heteroaryl(C₁-C₆)alkyl; R₂ is H, hydroxy, (C₁-C₆)alkoxy, (C₁-C₆)alkanoyloxy, NRₐR_{b} or SR_{c}; R₃ is H, aryl(C₁-C₆)alkyl, (C₁-C₆)alkyl, (C₁-C₆)alkanoyl, or (C₁-C₆)alkylC(═S); X is O, S, CH₂, or NY; Y is H, (C₁-C₆)alkyl, or aryl(C₁-C₆)alkyl; Rₓ, is a basic or positively charged group or an organic radical that comprises a basic or positively charged group; X is O, S, CH₂, or NY; Y is H, (C₁-C₆)alkyl, or aryl(C₁-C₆)alkyl; and Rₐ—R_{c}, are each independently H, (C₁-C₆)alkyl, (C₁-C₆)alkanoyl, phenyl, benzyl, phenethyl, or —C(═S)(C₁-C₆)alkyl; or a pharmaceutically acceptable salt thereof.

In a particular embodiment the invention provides a compound of formula (I), wherein R is halo, hydroxy, nitro, cyano, trifluoromethyl, trifluoromethoxy, (C₂-C₆)alkenyl, (C₂-C₆)alkynyl, (C₃-C₇)cycloalkyl, (C₅-C₇)cycloalkenyl, (C₃-C₆)cycloalkyl(C₁-C₆)alkyl, (C₅-C₇)cycloalkenyl(C₁-C₆)alkyl, aryl, heteroaryl, aryl(C₁-C₆)alkyl, heteroaryl(C₁-C₆)alkyl (C₁-C₆)alkoxy, (C₁-C₆)alkanoyloxy, NRₐR_{b} or SR_{c}.

In another particular embodiment the invention provides a compound of formula (I) wherein R is halo, hydroxy, nitro, cyano, trifluoromethyl, trifluoromethoxy, (C₃-C₇)cycloalkyl, (C₅-C₇)cycloalkenyl, (C₃-C₆)cycloalkyl(C₁-C₆)alkyl, (C₅-C₇)cycloalkenyl(C₁-C₆)alkyl, aryl, heteroaryl, aryl(C₁-C₆)alkyl, heteroaryl(C₁-C₆)alkyl (C₁-C₆)alkoxy, (C₁-C₆)alkanoyloxy, NₐR_{b} or SR_{c}.

In another particular embodiment, the invention provides the compound 5′-fluoro-6′-guanidino-17-(cyclopropylmethyl)-6,7-didehydro-4,5α-epoxy-3,14-hydroxyindolo-[2′,3′:6,7]morphinian; or 5′-fluoro6′-guanidino-17-(cyclopropylmethyl)-6,7-didehydro-4,5α-epoxy-3,14-hydroxyindolo-[2′, 3′: 6,7]morphinian; or a pharmaceutically acceptable salt thereof.

The invention also provides a novel compound of formula (I) wherein: R is hydrogen, halo, hydroxy, nitro, cyano, trifluoromethyl, trifluoromethoxy, (C₁-C₆)allyl, (C₂-C₆)alkenyl, (C₂-C₆)alkynyl, (C₃-C₇)cycloalkyl, (C₅-C₇)cycloalkenyl, (C₃-C₆)cycloalkyl(C₁-C₆)alkyl, (C₅-C₇)cycloalkenyl(C₁-C₆)allyl, aryl, heteroaryl, aryl(C₁-C₆)alkyl, heteroaryl(C₁-C₆)alkyl (C₁-C₆)alkoxy, (C₁-C₆)alkanoyloxy, NₐR_{b} or SR_{c}; R₁ is (C₁-C₆)alkyl, (C₂-C₆)alkenyl, (C₂-C₆)alkynyl, (C₃-C₇)cycloalkyl, (C₅-C₇)cycloalkenyl, (C₃-C₆)cycloalkyl(C₁-C₆)alkyl, (C₅-C₇)cycloalkenyl(C₁-C₆)alkyl, aryl, heteroaryl, aryl(C₁-C₆)alkyl, or heteroaryl(C₁-C₆)alkyl; R₂ is H, hydroxy, (C₁-C₆)alkoxy, (C₁-C₆)alkanoyloxy, NRₐR_{b} or SR_{c}; R₃ is H, aryl(C₁-C₆)alkyl, (C₁-C₆)alkyl, (C₁-C₆)alkanoyl, or (C₁-C₆)alkylC(═S); Rₓ is a basic or positively charged group or an organic radical that comprises a basic or positively charged group; X is O, S, CH₂, or NY; Y is H, (C₁-C₆)alkyl, or aryl(C₁-C₆)alkyl; and Rₐ—R_{c} are each independently H, (C₁-C₆)alkyl, (C₁-C₆)alkanoyl, phenyl, benzyl, phenethyl, or —C(═S)(C₁-C₆)alkyl; or a pharmaceutically acceptable salt thereof; wherein Rₓ is not CH₂)ₙ—NH—C(═R₄)—R₅—R₆, wherein n is 0, 1, 2, 3, 4; R₄ is ═O, ═S, or ═NR_{d}; R_{d} is H, CN, CONH₂, COCF₃, (C₁-C₆)alkanoyl, (C₁-C₆)alkyl, or (CH₂)ₚNRₑR_{f}, or R_{d} together with R₆ is —(CH₂)_{q}— and forms a ring; p is 1, 2, 3, or 4; R₅ is NRₘ; R₆ is H, (C₁-C₆)alkyl, (C₃-C₇)cycloalkyl, aryl, heteroaryl, aryl(C₁-C₆)alkyl, heteroaryl(C₁-C₆)alkyl, NR_{g}Rₕ(C₁-C₆)alkyl, or C(═NRⱼ)NHRₖ; or when R₄ is ═NR_{d}, R₆ together with R_{d} is —(CH₂)_{q}— and forms a ring; q is 2 or 3; Rₑ—R_{f} are each independently H, (C₁-C₆)alkyl, (C₁-C₆)alkanoyl, phenyl, benzyl, phenethyl, or —C(═S)(C₁-C₆)alkyl; R_{g} and Rₕ are each independently H, (C₁-C₆)alkyl, (C₁-C₆)alkanoyl, —C(═NH)NRₐR_{b}, or —C(═S)(C₁-C₆)alkyl, or R_{g} and Rₕ together with the nitrogen to which they are attached are pyrrolidino, piperidino or morpholino; Rⱼ and Rₖ are each independently H, (C₁-C₆)alkyl, (C₂-C₆)alkenyl, (C₂-C₆)alkynyl, (C₃-C₇)cycloalkyl, (C₃-C₇)cycloalkyl(C₁-C₆)alkyl, (C₅-C₇)cycloalkenylalkyl, aryl, heteroaryl, aryl(C₁-C₆)alkyl, or heteroaryl(C₁-C₆)alkyl; and Rₘ is hydrogen or (C₁-C₆)alkyl.

The invention also provides a pharmaceutical composition comprising a compound of formula (I) or (II), and a pharmaceutically acceptable carrier.

It will be appreciated by those skilled in the art that compounds of formula (I) or (II) having a chiral center in the group Rₓ in formula (I) or in the groups NHC(═R₄)R₅R₆ in formula (II) may exist in and be isolated in optically active and racemic forms. Some compounds may exhibit polymorphism. It is to be understood that the methods of the present invention can be practiced with any racemic, optically-active, polymorphic, or stereoisomeric form, or mixtures thereof, of a compound of the invention, which possess the selective pharmacological properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine selective agonist activity using the tests described herein, or using other similar tests which are known in the art.

The following definitions are used, unless otherwise described: halo is fluoro, chloro, bromo, or iodo. Alkyl, alkoxy, etc. denote both straight and branched groups; but reference to an individual radical such as “propyl” embraces only the straight chain radical, a branched chain isomer such as “isopropyl” being specifically referred to. Aryl denotes a phenyl radical or an ortho-fused bicyclic carbocyclic radical having about nine to ten ring atoms in which at least one ring is aromatic. Heteroaryl encompasses a radical of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto.

Specific values listed below for radicals, substituents, and ranges, are for illustration only; they do not exclude other defined values or other values within defined ranges for the radicals and substituents.

Specifically, (C₁-C₆)alkyl can be methyl, ethyl, propyl, isopropyl, butyl, iso-butyl, sec-butyl,pentyl, 3-pentyl, or hexyl; (C₃-C₇)Cycloalkyl can be cyclopropyl, cyclobutyl, cyclopentyl, cyclohexyl, or cycloheptyl; (C₃-C₇)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be acetyl, propanoyl or butanoyl; and (C₂-C₆)alkanoyloxy can be acetoxy, propanoyloxy, butanoyloxy, isobutanoyloxy, pentanoyloxy, or hexanoyloxy; aryl can be phenyl, indenyl, or naphthyl; heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

Preferably, in compounds of formulas (I) or (II) R is hydrogen or halo.

Preferably, R is at the 5′-position.

Specifically, R₁ is (C₂-C₆)alkenyl or (C₃-C₆)Cycloalkyl(C₁-C₆)alkyl.

More specifically, R₁ is cyclopropylmethyl or allyl.

Specifically, R₂ is OH.

Specifically, R₃ is H.

Specifically, R₄ is ═NR_{d}. More specifically, R₄ is ═NH or ═NCN.

Specifically, R₅ is NH.

Specifically, R₆ is hydrogen, methyl, ethyl, propyl, butyl, pentyl, hexyl, 3-(dimethylamino)-propyl, or 2-pyrrolidinoethyl. More specifically, R₆ is H. Another specific R₆ is C(═NRⱼ)NHRₖ.

Specifically, Rₘ is hydrogen.

Specifically, n is 0.

Specifically, n is 1.

Specifically, X is CH₂ or NH. More specifically, X is CH₂. More specifically X is NH.

Preferred compounds for use in the methods of the invention possess the core ring structure of formula (I) and are substituted at the 6′ position with a group that is positively charged or that is capable of being positively charged under physiological conditions in a target tissue (i.e. a basic or positively charged group). Thus, in a compound of formula (I), the group Rₓ is preferably a basic or positively charged group or an organic radical that comprises a basic or positively charged group. More preferably, Rₓ is an organic radical that comprises a basic or positively charged group that is spatially oriented similarly to the basic or positively charged group in compound 7. Preferred basic or positively charged groups include quaternary amines, or other amines that can form positively charged ammonium salts under physiological conditions. For example, Rₓ can be an organic group comprising a mono-, di-, tri-or tetra- substituted amine group, wherein the amine group is separated from the 6′-carbon in formula (I) by from about 5 to about 100 Angstroms. Preferably, the amine group is separated from the 6′-carbon in formula (I) by from about 5 to about 30 Angstroms.

A specific compound of formula (I) is a compound wherein: R₁ is (C₁-C₆)alkyl, (C₂-C₆)alkenyl, (C₂-C₆)alkynyl, (C₃-C₇)cycloalkyl, (C₅-C₇)cycloalkenyl, (C₃-C₆)cycloalkyl(C₁-C₆)alkyl, (C₅-C₇)cycloalkenyl(C₁-C₆)alkyl, aryl, heteroaryl, aryl(C₁-C₆)alkyl, heteroaryl(C₁-C₆)alkyl; R₂ is H, OH, (C₁-C₆)alkoxy, (C₁-C₆)alkanoyloxy, NRₐR_{b} or SR_{c}; R₃ is H, aryl(C₁-C₆)alkyl, (C₁-C₆)alkyl, (C₁-C₆)alkanoyl, or (C₁-C₆)alkylC(═S); R₄ is ═O, ═S, ═NR_{d}, wherein R_{d} is H, CN, CONH₂, COCF₃, (C₁-C₆)alkanoyl, (C₁-C₆)alkyl, or (CH₂)ₚNRₑR_{f}, wherein p=1-4; R₅ is NH; R₆ is H, (C₁-C₆)alkyl, (C₃-C₇)cycloalkyl, aryl, heteroaryl, aryl(C₁-C₆)alkyl, heteroaryl(C₁-C₆)alkyl, NR_{g}Rₕ(C₁-C₆)alkyl, or C(═NRⱼ)NHRₖ; or when R₄ is ═N, R₆ can be —CH₂)_{q}— and form a ring with the N of R₄, wherein q is 2 or 3; X is O, S, or NY, wherein Y is H (C₁-C₆)alkyl, or aryl(C₁-C₆)alkyl; n is 0, 1, 2, 3, or 4; Rₐ—R_{f} are each independently H, (C₁-C₆)alkyl, (C₁-C₆)alkanoyl, or —C₆)alkyl; and C(═S)(C₁-C₆)alkyl; R_{g} and Rₕ are each independently H, (C₁-C₆)allyl, (C₁-C₆)alkanoyl, —C(═NH)NRₐR_{b}, or —C(═S)(C₁-C₆)alkyl, or R_{g} and Rₕ together with the nitrogen to which they are attached are pyrrolidino, piperidino or morpholino; and Rⱼ and Rₖ are each independently H, (C₁-C₆)alkyl, (C₂-C₆)alkenyl, (C₂-C₆)alkynyl, (C₃-C₇)cycloalkyl, (C₃-C₇)cycloalkyl(C₁-C₆)alkyl, (C₅-C₇)cycloalkenylalkyl, aryl, heteroaryl, aryl(C₁-C₆)alkyl, or heteroaryl(C₁-C₆)alkyl; or a pharmaceutically acceptable salt thereof.

A specific compound of formula (I) is a compound wherein R₆ is not (C₁-C₆)alkyl when n is 1, R₄ is NH, and R₅ is NH.

A specific compound of formula (I) is a compound wherein R_{d} together with R₆ is —(CH₂)_{q}— and forms a ring.

A specific compound of formula (I) is 6′-guanidinyl-17-cyclopropylmethyl-6,7-didehydro-4,5α-epoxy-3,14-dihydroxyindolo[2′,3′:6,7]morphinan or a pharmaceutically acceptable salt thereof (e.g., ditrifluoroacetate dihydrate).

A specific compound of formula (I) is 6′-N-methylguanidinyl-17-cyclopropylmethyl-6,7-didehydro-4,5-α-epoxy-3,14-dihydroxyindolo[2′,3′:6,7]morphinan or a pharmaceutically acceptable salt thereof (e.g., ditrifluoroacetate dihydrate).

A specific compound of formula (I) is 6′-N-ethylguanidinyl-17-cyclopropylmethyl-6,7-didehydro-4,5-α-epoxy-3,14-dihydroxyindolo[2′,3′:6,7]morphinan or a pharmaceutically acceptable salt thereof (e.g., ditrifluoroacetate dihydrate).

A specific compound of formula (I) is 6′-N-propylguanidinyl-17-cyclopropylmethyl-6,7-didehydro-4,5-α-epoxy-3,14-dihydroxyindolo[2′,3′:6,7]morphinan or a pharmaceutically acceptable salt thereof (e.g., ditrifluoroacetate dihydrate).

A specific compound of formula (I) is 6′-N-butylguanidinyl-17-cyclopropylmethyl-6,7-didehydro-4,5-α-epoxy-3,14-dihydroxyindolo[2′,3′:6,7]morphinan or a pharmaceutically acceptable salt thereof (e.g., ditrifluoroacetate dihydrate).

A specific compound of formula (I) is 6′-N-pentylguanidinyl-17-cyclopropylmethyl-6,7-didehydro-4,5α-epoxy-3,14-dihydroxyindolo[2′,3′:6,7]morphinan or a pharmaceutically acceptable salt thereof (e.g., ditrifluoroacetate dihydrate).

A specific compound of formula (I) is 6′-N′-cyano-N′-[17-(cyclopropylmethyl)-6,7-didehydro-4,5α-epoxy-3,14-dihydroxyindolo[2′,3′:6,7]morphinian]-guanidine, or a pharmaceutically acceptable salt thereof.

A specific compound of formula (I) is 6′-N-cyano-N′-[3-(dimethylaminopropyl)]-N″-[17-(cyclopropylmethyl)-6,7-didehydro-4,5α-epoxy-3,14-dihydroxyindolo[2′,3′:6,7]morphinian]-guanidine, or a pharmaceutically acceptable salt thereof.

A specific compound of formula (I) is 6′-N-cyano-N′-[2-(1-aminoethylpyrrolidine)]-N″-[17-(cyclopropylmethyl)-6,7-didehydro-4,5α-epoxy-3,14-dihydroxyindolo[2′,3′:6,7]morphinian]-guanidine, or a pharmaceutically acceptable salt thereof.

A specific compound of formula (I) is 5′-Fluoro-6′-guanidino-17 -(cyclopropylmethyl)-6,7-didehydro-4,5α-epoxy-3,14-hydroxyindolo-[2′,3′:6,7]morphinian (23a), or a pharmaceutically acceptable salt thereof.

A specific compound of formula (I) is 5′-Chloro-6′-guanidino-17-(cyclopropylmethyl)-6,7-didehydro-4,5α-epoxy-3,14-hydroxyindolo-[2′,3′:6,7]morphinian (23b), or a pharmaceutically acceptable salt thereof.

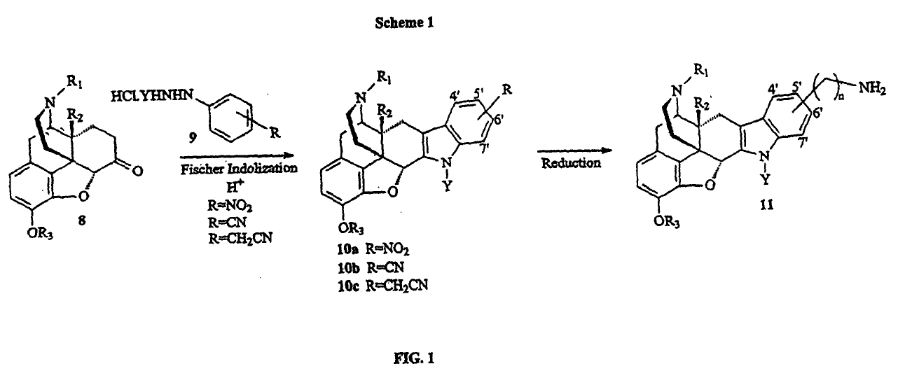

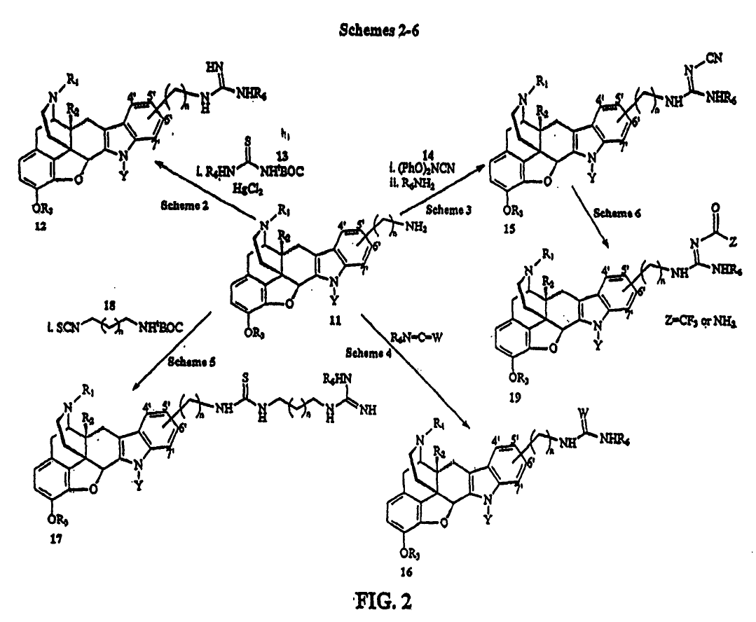

Compounds of formula (I) and salts or solvates thereof, may be prepared by the methods illustrated in Schemes 1-6 (FIGS. 1 and 2), or by modification thereof, using readily available starting materials, reagents and conventional synthetic procedures.

The compounds of general formula (I) wherein X is NH can be readily synthesized by reaction of a 4,5-epoxy-6-ketomorphinan such as naltrexone (8, R₁=cyclopropylmethyl=CPM, R₂═OH, R₃═H, scheme 1) with a substituted phenyl hydrazine 9 under Fischer indolization conditions (see D. L. Hughes. Org. Prep. Proc. Intl. 25(6), 607-632, 1993). The indolomorphinan products 10 are subsequently reduced to the primary amines 11 by utilizing the reduction conditions set out in FIG. 1 (Scheme 1).

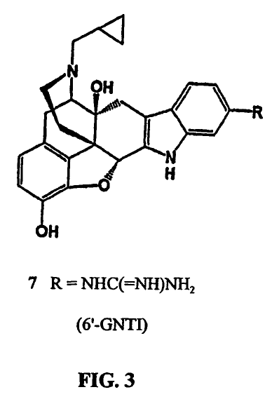

Guanidinyl compounds of general formula 12 (FIG. 2, scheme 2) can be prepared from amines 11 (where n=0-3) by reaction with a modified thiourea derivative 13 using mercuric(II)chloride assisted guanidylation protocols (see K Y. Kim; L. Qian. Tet. Lett. 1993, 34, 48, 7677-7680 and M. A. Poss; E. Iwanowicz; J. A. Reid; J. Lin; Z. Gu. Tet. Lett. 33, 40, 5933-5936, 1992) followed by acid deprotection. 6′-GNTI (7, FIG. 3)(R=hydrogen, R₁=cyclopropylmethyl=CPM, R₂═OH, R₃═H, X═NH, R₄═NH, R₅═NH, R₆═H as its trifluoroacetate salt, general formula (I)) or more specifically 6′-guanidinyl-17-cyclopropylmethyl-6,7-didehydro-4,5-αepoxy-3,14-dihydroxyindolo[2′,3′:6,7]-morphinan ditrifluoroacetate dihydrate is a specific example of this class.

Cyanoguanidines of general formula 15 (FIG. 2, scheme 3) maybe obtained from amines 11 by reaction with diphenyl-N-cyanocarbonimidate 14 (see C. J. Durant et al. J. Med. Chem. 1977, 20, 7, 901 and R. L. Webb, C. S. Labaw. J. Het. Chem. 19, 1205, 1982) followed by displacement of phenol from the intermediate by reaction with a primary amine or general formula R₆NH₂. 6′-CNGNTI (R=hydrogen, R₁=cyclopropylmethyl=CPM, R₂═OH, R₃═H, X═NH, R₄═NCN, R₅═NH, R₆═H, general formula (I)) or more specifically 5′-N-cyanoguanidinyl-17-cyclopropylmethyl-6,7-didehydro-4,5-α-epoxy-3,14-dihydroxyindolo[2′,3′:6,7]-morphinan is a specific example of this class.

Ureas of general formula 16 (FIG. 2, scheme 4) wherein W is O or S can be readily prepared by reaction of amines 11 with R₆NCW. Specific variants of the above are cited in reaction Scheme 5. Commercially available modified isothiocyanates of general formula 18 (n=0-3) (Fluka) are reacted with amines 11. Deprotection of the terminal tert-BOC moiety followed by guanidylation (see K. Y. Kim; L. Qian. Tet. Lett. 1993, 34, 48, 7677-7680 and M. A. Poss; E. Iwanowicz; J. A. Reid; J. Lin; Z. Gu. Tet. Lett. 33, 40, 5933-5936, 1992) and a second acid mediated tert-BOC deprotection yields compounds of general formula 17.

Cyanoguanidines 15 maybe modified further as depicted in FIG. 2, Scheme 6 to afford compounds of general formula 19 (see S. N. Thorn. Tet. vol 49, 31, 6885, 1993). Compounds of formula (I) wherein X is CH₂, O or Scan be prepared from intermediates structurally similar to 11 wherein NY has been replaced by CH₂, O, or S. These intermediates can be prepared as generally disclosed in U.S. Pat. No. 4,816,586, which is incorporated by reference herein, which also discloses methods suitable for the preparation of salts of compounds of formula (I).

4,5-Epoxy-6-ketomorphinans of general structure 8 (FIG. 1, scheme 1) can be prepared by synthetic methods that are well known in the art of organic chemistry (see U.S. Pat. No. 5,457,208 and citations therein).

In cases where compounds are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compounds of formula (I) can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula (I) to the skin are known to the art; for example, see Jacquet et al. (U.S. Pat. No. 4,608,392), Geria (U.S. Pat. No. 4,992,478), Smith et al. U.S. Pat. No. 4,559,157) and Wortzman (U.S. Pat. No. 4,820,508).

Useful dosages of the compounds of formula (I) can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose can be, for example, in the range of from about 0.01 to about 10 mg/kg, e.g., preferably from about 0.05 to about 1.0 mg/kg of body weight per day, most preferably in the range of 0.1 to 0.5 mg/kg/day.

The compounds of formula (I) can conveniently administered, for example, in unit dosage form; for example, containing 1 to 50 mg, conveniently 2 to 20 mg, most conveniently, 5 to 15 mg of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The ability of a compound to selectively modulate the activity of the delta-kappa opioid receptor can be determined using pharmacological models that are well known to the art, or using the procedures described below.

Mouse Vas Deferens

Mouse vasa deferentia are prepared using the method of Henderson, et al (see Henderson, G.; Hughes, J.; Kosterlitz, H. W., Br. J. Pharmacol. 1972, 46, 764-766). Male ICR mice (30-35 g) are killed by cervical dislocation. Both vasa deferentia are removed and mounted between platinum ring electrodes and placed in a 10-mL organ bath containing a modified Kreb's solution (NaCl, 118 mM; KCl, 4.70 mM;CaCl₂, 2.52 mM; KH₂PO₄, 1.19 mM; NaHCO₃, 25 mM; glucose, 11.48 mM; pH =7.4) at 37° C. The bath is continuously bubbled with a 95% O₂, 5% CO₂, gas mixture. One end of the vas deferens is attached to the electrode assembly, the other is attached to a Statham-Gould UC-3 isometric force transducer using 6.0 surgical silk. The vasa deferentia are stimulated transmurally with a Grass S44 stimulator (square waves of supramaximal voltage (70 V) for 1 msec and a frequency of 0.1 Hz). Resting tension is 200 mg. Vasa deferentia are stimulated continuously for 20 minutes before each experiment to allow equilibration to occur. The tissues are washed every 10 minutes during this period.

Guinea Pig Ileal Longitudinal Muscle

Ilea are prepared using the method of Rang (see Rang, H. P., Br. J. Pharmacol. 1964, 22, 356-365). Male Dunkin-Hartley guinea pigs (350-400 g) are killed by CO₂ inhalation. The ilea are taken approximately 10 cm from the ileocaecal junction and placed in a modified Kreb's solution (NaCl, 118 mM; KCl, 4.70 mM;CaCl₂, 2.52 mM; KH₂PO₄, 1.19 mM; MgSO₄, 1.19 MM; NaHCO₃, 25 mM; glucose, 11.48 mM; chlopheniramine maleate, 1.25 μM; pH=7.4) at room temperature. A 1 cm strip of longitudinal muscle with the myenteric plexus attached is dissected and mounted between platinum electrodes and placed in a 10-mL organ bath containing the Kreb's solution at 37° C. and continuously bubbled with a 95% O₂ and 5% CO₂ gas mixture. One end of the muscle strip is attached to the electrode assembly, the other is attached to Statham-Gould UC-3 isometric force transducer using 3.0 surgical silk. Ilea are stimulated transmurally with a Grass S44 stimulator (square waves of supramaximal voltage (80 V) for 0.5-msec duration and a frequency of 0.1 Hz). Resting tension is 1 g. Guinea pig ilea are stimulated continuously for 90 minutes before each experiment to allow equilibration to occur. Tissues are washed every 30 minutes during this period.

Radioligand Binding

Compounds can be screened for binding to stable cell lines of human embryonic kidney (HEK) or Chinese hamster ovary (CHO) cells which express μκ, or δ opioid receptors. The cells are grown in tissue culture plates in Dulbecco's Modifed Eagle Medium (DMEM) containing 10% fetal calf serum (Hyclone), 1% Penicillin-Streptomycin (5000 units/ml each in 0.85% saline; GIBCOBRL), and 0.5% Geneticin (50 mg/ml; GIBCOBRL). Cells are grown in a humidified CO₂ incubator at 37° C. until confluent. Media is changed as necessary. At confluency, the media is removed and 12 mL of PBS/EDTA (2.92 g NaCl, 0.69 g NaH₂PO₄.H₂O, and 0.20 g EDTA (free acid) in 500 mL water, pH 7.5, pre-warmed to 37° C. is added and the cells are pipetted into sterile centrifuge tubes and centrifuged at 1000 rpm for 5 min. using an EEC Centra CL3R centrifuge. The supernatant is discarded and the cells are re-suspended in 25 mM HEPES buffer (pH=7.40) (12-15 mL/100 mm2 plate) and placed on ice until used. Radioligand competition assays are performed by adding varying concentrations of compound (typically from 0.1 nM to 1000 nM) to duplicate tubes containing 0.1 nM ³[H]diprenorphine, 100-500 μg protein (400 μL cell suspension), and 25 mM HEPES buffer (pH=7.40) to make a final volume of 0.5 mL. Incubation is at room temperature for 90 minutes, after which the reaction is terminated by filtering the cells through Whatmann GF/C filter paper, pre-soaked in 0.25% polyethylenimine in distilled water), using a Brandell M-48 cell harvester. The trapped cells are rinsed three times with 4 mL ice cold HEPES buffer. Filter papers are placed in scintillation vials, immersed in 4 mL Ecolite+Scintillation cocktail (ICN), and counted in a Beckmann LS 3801 scintillation counter. Non-specific binding is determined using 10 μM naloxone. Mean IC₅₀ and Kᵢ values are obtained from at least three different experiments. Data for individual experiments are analyzed using RADLIG and LIGAND (Biosoft). Kᵢ values are calculated using K_{D} values obtained by conducting ³[H]diprenorphine saturation curves on each cell line. These assays are performed as above, but with varying concentrations (typically 30 -3000 pM) of radioligand. For each concentration, the non-specific binding is determined as above and total radioactivity added is measured. K_{D} values are obtained by analyzing data using RADLIG and LIGAND.

By these methods, it can be determined whether a compound binds to a delta-kappa receptor, e.g., at least 3-fold more strongly than it binds to a kappa receptor. The K_{D} values can be determined for binding to cells expressing delta and kappa receptors, and optionally other receptor subunits, and compared to the K_{D} values determined for binding to cells expressing only the kappa receptor, to quantify how much more or less strongly a compound binds to the delta-kappa receptor than to the kappa receptor.

Tail Flick

In the tail-flick assay which was modified for mice, the animal responses are made quantal by establishing and end point at the mean peak effect which represents an increase in the reaction time of an individual animal of greater than three S.D. of the control mean reaction time for all animals used in the group (see F. E. d'Amour et al., J. Pharmacol. Exp. Ther., 1941, 72, 7479; and J. J. Rady et al., J. Pharmacol Exp. Ther., 2000, 224, 93-101). Non-responding animals will be removed from the heat stimulus when reaction times exceed 3 seconds to avoid damage to their tails. At least 50 animals will be used to determine each peak time and ED₅₀ dose-response curve. The ED₅₀ and its 95% confidence interval are estimated using computer programs for both of these statistical procedures.

Results

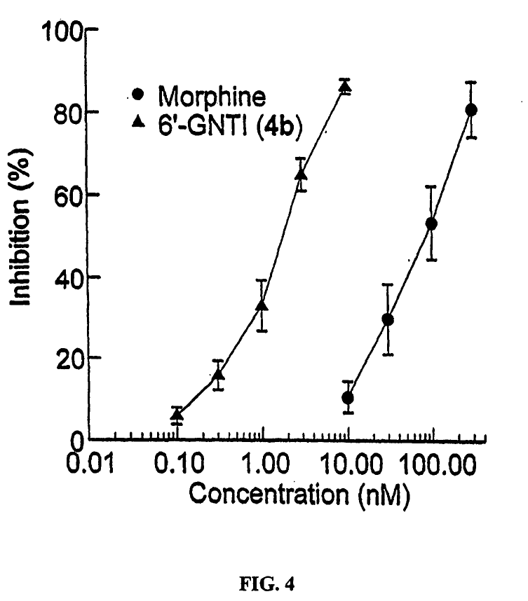

The strength of muscular contraction in response to electrical stimulation of guinea pig ilium was measured with bathing of the ilium in control medium or medium containing morphine or compound 7. FIG. 4 shows the extent of inhibition of the strength of contraction induced by compound 7 and morphine. In the guinea pig ilium preparation compound 7 is 50× more potent than morphine (FIG. 4) and is competitively antagonized by the selective kappa antagonist, nor-BNI. Significantly, it is non-competetively antagonized by the delta antagonist, naltrindole (NTI).

In the mouse vas deferens preparation (MVD), compound 7 is inactive as an agonist or as an antagonist. These data suggest that the agonist effect is mediated through binding of compound 7 to one monomeric subunit of the heterodimer. Antagonism can be effected either by binding of NTI to the second subunit through an allosteric effect or by norBNI-induced competitive antagonism at the recognition site that binds compound 7. In this connection, additional receptor binding studies showed that compound 7 is selective for cloned kappa opioid receptors.

The above results suggest coupling between delta and kappa receptors that are associated as heterodimers. The GPI is well-known to be responsive to mu and kappa agonists. The previous reports that the GPI contains cryptic delta receptors that do not mediate a delta agonist effect, together with the above data, suggest that the inactivity of the delta receptor is due to its dimerization with the kappa receptor.

Intrathecal (i.t.) administration of compound 7 afforded strong analgesia (ED₅₀=0.45 nmol/mouse) which could be antagonized by norBNI. However, intraventricular (icv) administration of compound 7 at 10-fold higher concentration produced no analgesic effect. Only when the concentration was 20-fold that of the i.t. dose was a weak effect (20% analgesia) observed. It is noteworthy that compound 7 appears to exert weak antagonism of the kappa-selective agonist, U50488, when they are co-administered icv. Thus, compound 7 may function as an antagonist or partial agonist at monomeric or homodimeric opioid receptors in the brain.

The molecular basis for the selectivity of compounds of formula (I) for the kappa-delta heterodimer is believed to result from the presence of a positively charged moiety at the 6′-position, which is available for ion paring with the negatively charged glutamate-297 of the kappa opioid receptor. Thus, opioid ligands that contain such a positively charged group in a position similar to that of compound 7 are expected to have qualitatively similar biological activity.

These results demonstrate a clear separation of analgesic potency between the spinal cord and the brain, possibly as a result of the different conformational states between kappa-delta heterodimers and monomeric or homodimeric kappa opioid receptors. Compound 7 as well as other ligands that are selective for such heterodimers (e.g. compounds of formula (I)) are potentially useful as analgesics. They should not exhibit many of the undesirable side-effects that accompany activation of opioid receptors in the brain.

Preferred compounds for use in the methods of the invention bind to a delta-kappa opioid receptor at least 3, 5, 10, or 50 fold more strongly than they bind to a kappa receptor. Preferred compounds for use in the methods of the invention also produce an agonist effect at the delta-kappa receptor that is at least about 3, 5, 10, or 50 times greater than the agonist effect at the kappa receptor. Preferred compounds for use in the methods of the invention also produce an agonist effect at opioid receptors outside the brain that is at least about 3, 5, 10, or 50 times greater than the agonist effect at the kappa receptor.

Preferred compound for use in the methods of the invention bind to a delta-kappa opioid receptor at least 3, 5, 10, or 50 fold more strongly than they bind to a delta receptor. Preferred compounds for use in the methods of the invention also produce an agonist effect at the delta-kappa receptor that is at least about 3, 5, 10, or 50 times greater than the agonist effect at the delta receptor. Preferred compounds for use in the methods of the invention also produce an agonist effect at opioid receptors outside the brain that is at least about 3, 5, 10, or 50 times greater than the agonist effect at the delta receptor.

Preferred compound for use in the methods of the invention binds to a delta-kappa opioid receptor at least 3, 5, 10, or 50 fold more strongly than it binds to a mu receptor. Preferred compounds for use in the methods of the invention also produce an agonist effect at the delta-kappa receptor that is at least about 3, 5, 10, or 50 times greater than the agonist effect at the mu receptor. Preferred compounds for use in the methods of the invention also produce an agonist effect at opioid receptors outside the brain that is at least about 3, 5, 10, or 50 times greater than the effect at the mu receptor.

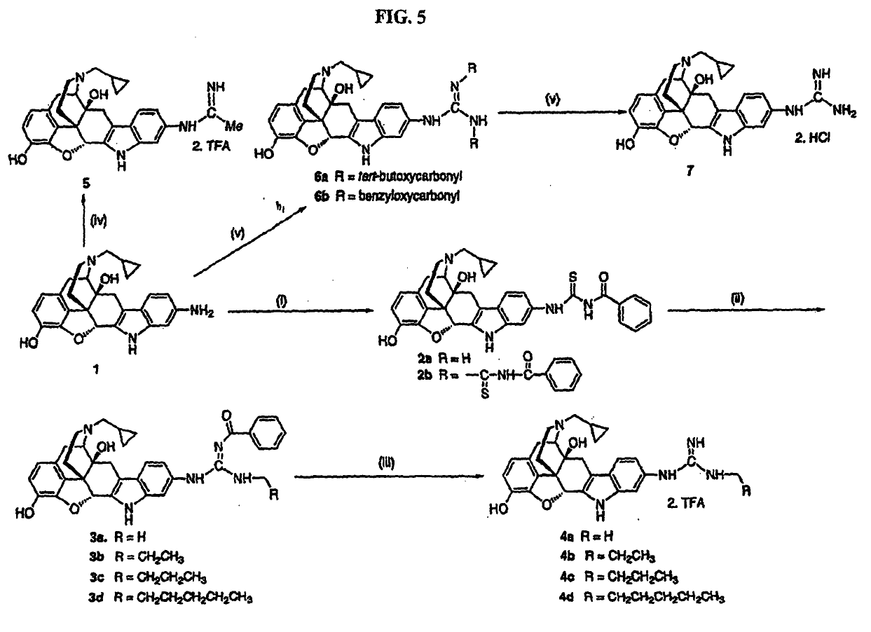

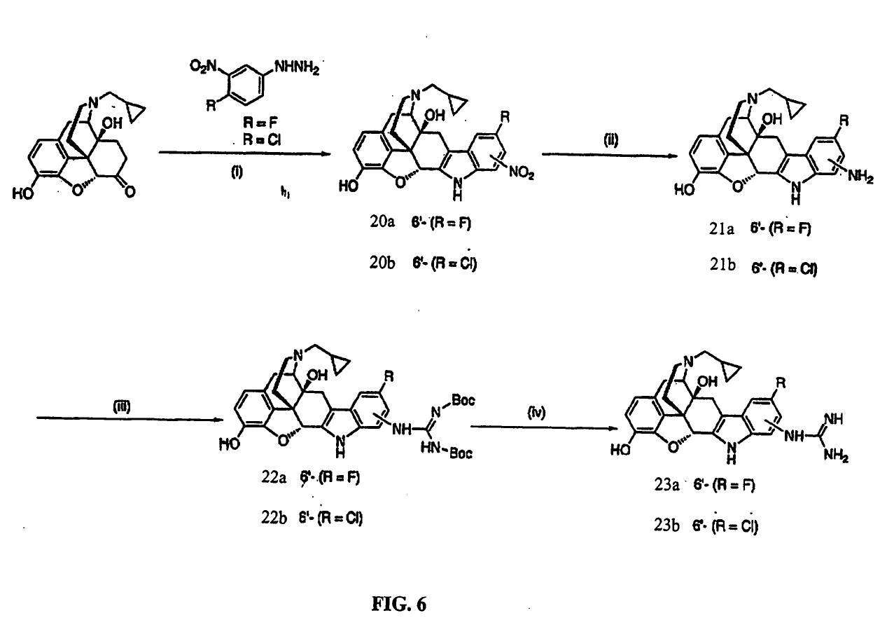

FIGS. 5 and 6 show representative syntheses of compounds of the invention. The reagents in FIG. 5 for step (i) are CH2Cl2/benzoylisothiocyanate; for step (ii) are HgCl₂/NEt₃/CH₂Cl₂/R—CH₂—NH₂; for step (iii) are K₂CO₃/MeOH, room temperature, 3 days; for step (iv) are ethyoxyacetamidoacetate/EtOH/refluxed 18 hours; for step (v) are HgCl₂/NEt₃/CH₂Cl₂/R—NH(═NR)C—SMe; for step (vi) are 10% Pd/C/H₂/MeOH, 75 psi or 2N HCl in MeCO₂Et. The reagents in FIG. 6 for step (i) are AcOH/conc. HCl (4:1), 2 days; for step (ii) are Raney Nickel/NH₂NH₂ H₂O/ethanol, 2 hours; for step (iii) are HgCl₂/NEt₃/CH₂Cl₂/Boc-NH(═NBoc)C—SMe; for step (vi) are TFA/CH₂Cl₂.

The invention is further illustrated by the following non-limiting Examples. The preparation of representative compounds of formula (I) is illustrated by Examples 1 and 2. Further data evidencing delta-kappa receptors in the spine is presented in Example 3.

### EXAMPLE 1

6′-N′-(N″,N′″-Bis(tert-butoxycarbonyl)guanidino-17-(cyclopropylmethyl)-6,7didehydro-4,5α-epoxy-3,14-hydroxyindolo-[2′,3′:6,7]morpbinian (6a). A mixture of compound 1 (FIG. 5) (216 mg, 0.5 mmol), HgCl₂ (250 mg, 0.83 mmol), 1,3-bis(tert-butoxycarbonyl)-2-methyl-2-thiopseudourea (200 mg, 0.7 mmol) in freshly dried CH₂Cl₂ containing few drops of NEt₃ was allowed to stir for 18 h under a sealed N₂.atmosphere at room temperature. After completion of the reaction (24 hours) the mixture was filtered trough Celite under vacuum to remove mercuric sulfide , and the residue was washed thoroughly with methanol. The combined filtrate was concentrated to give a solid product. This gave two products which were separated by column chromatography (CH₂Cl₂-MeOH—NH₄OH, 94.5:5.0:0.5); the major product was compound 6a (260 mg, 78%): mp 270° C. (dec); ¹H NMR (DMSO-d₆): δ 11.43 (s, 1H, NH), 11.20 (s, 1H, NH), 10.00 (s, 1H, NH), 8.88 (s, 1H, Ar—OH), 7.69 (s, 1H, ArH), 7.25 (d, 1H, J=8.7 Hz, ArH), 6.90 (d, 1H, J=8.7 Hz, ArH), 6.47 (d, 1H, J=8.1 Hz, ArH), 6.42 (d, 1H, J=8.1 Hz, ArH), 5.44 (s, 1H, 5-H), 4.70 (b, 1H, 14-OH), 3.24-3.07 (m, 2H), 3.02 (m, 1H), 2.60 (m, 2H), 2.48-2.08 (m, 5H), 1.53 (m, 1H), 1.48 (s, 9H, ^{t}Bu), 1.38 (s, 9H, ^{t}Bu), 0.80 (m, 1H), 0.45 (m, 2H), 0.12 (m, 2H); ¹³C NMR (DMSO-d₆): δ 153.35, 152.81, 143.56, 140.31, 136.98, 131.64, 131.44, 130.87, 124.73, 124.24, 118.73, 117.37, 114.99, 110.56, 106.23, 84.34, 72.66, 62.11, 59.11, 47.74, 43.80, 39.15, 31.79, 29.18, 28.40, 23.14, 9.71, 4.35, 3.97. HRMS (FAB) m/z 672.3405 (M+H)⁺, C₃₇H₄₅N₅O₇ requires 671.3397.

### EXAMPLE 2

6′-Guanidino-17-(cyclopropylmethyl)-6,7-didehydro-4,5α-epoxy-3,14-hydroxyindolo-[2′,3′:6,7]morphinian (7). Compound 6a (500 mg, 0.75 mmol) was dissolved in a mixture of TFA (3.0 mL) and dried CH₂Cl₂ (28 mL) and allowed to stir under N₂ atmosphere at room temperature for 36 h. The reaction was monitored by TLC, and after 36 hours, CH₂Cl₂ and TFA were removed with a stream of N₂, leaving a residue which was sujected to column chromatography (CH₂Cl₂-MeOH—NH₄OH, 78:20:2) to give 7. Further purification was accomplished by preparative TLC to give 7 (260 mg, 74%) as a free base; IR KBr disk v (cm⁻¹): 3450-3150 (br), 1683 (s), 1506, 1463, 1433, 1330, 1202, 1132; ¹H NMR(DMSO₆): δ 11.50(s, 1H, NH), 9.96 (s, 1H, NH), 9.29 (s, 1H, NH), 8.95 (s, 1H, Ar—OH), 7.36-7.09 (m, 3H, ArH and NH₂),6.77 (d, 1H, J=8.10 Hz, ArH), 6.59-6.52 (m, 2H, ArH), 6.39 (s, 1H), 5.67 (s, 1H, 5-H), 4.05 (b, 1H, 14-OH), 3.43-3.23 (m, 3H), 3.18-3.06 (m, 2H), 2.96-2.91 (m, 2H), 2.68-2.57 (m, 2H), 2.50 (m, 1H), 1.78 (d, 1H, J=11.7 Hz), 1.05 (m, 1H), 0.68 (m, 1H), 0.58 (m, 1H), 0.40 (m, 2H). HRMS (FAB) m/z 472.2356 (M+H)⁺, C₂₇H₂₉N₅O₃ requires 471.2270.

### EXAMPLE 3

Methods

In this Example antagonism against antinociception caused by certain known selective opioid agonists was tested. Antagonism against each agonist was tested with (1) the κ antagonist norbinaltorphimine (nor-BNI), (2) the δ₁antagonist 7-benzylidenenaltrexone (BNTX), (3) the δ₂ antagonist naltriben (NTB), and (4) the μ antagonist D-Phe-Cys-Tyr-D-Trp-Orn-Thr-Phe-Thr-NH₂ (CTOP).

The selective opioid agonist antinociceptive agents tested were [D-Ala²,N-Me-Phe⁴,Gly-ol⁵]enkephalin (DAMGO); [D-Pen^{2,5}]enkephalin (DPDPE); [D-Ala², Glu⁴]deltorphin (Deltorphin II); and 3,4-dichloro-N-methyl-N-[2-(1-pyrrolidiyl)cyclohexyl]benzeneacetamide (U50488).

The antinociceptive agents were intrathecally injected into mice. Male CD1 mice (Harlan Sprague Dawley) weighing between 20-25 grams were used. They were housed at least 24 hr before the experiment in a temperature controlled (23° C.) room. Each animal was used only once. A modified tail flick assay (Tulunay, F. C., and Takemori, A. E. (1974) J. Pharmnacol. Exp. Ther. 190:395-400 and Tulunay, F. C., and Takemori, A. E. (1974) J. Pharmacol. Exp. Ther. 190:395-400) was used for the analgesic assay. At least three groups of 10 mice were used to generate dose-response curves. A mouse was regarded as positive for antinociception if the latency to flick its tail was more than the control latencies plus 3 S.D. of the mean of the reaction time of the group. The reaction times were determined at the peak time for antinociception of the combined agonist and antagonist.

The effective dose at which 50% of the experimental animals respond to the stimulus (ED₅₀) (nmol/mouse) for each agent was determined in the absence of any antagonists, and in the presence of each of the antagonists. The antagonists were administered at the following doses: 2.5 nmol/mouse nor-BNI, 25 pmol/mouse BNTX, 50 pmol/mouse NTB, and 5.9 pmol/mouse CTOP. The potency ratio, i.e., the ED₅₀ in the presence of the antagonist / the ED₅₀ in the absence of any antagonist, was calculated.

A potency ratio of approximately 1 indicates that the antinociceptive agent was not inhibited by the particular antagonist tested, and therefore suggests that the antinociceptive agent does not act in the spine by binding to a complex containing the receptor type to which the antagonist binds.

The animal studies used in these experiments were approved by the University of Minnesota Institutional Animal Care and Use Committee (IACUC).

Results

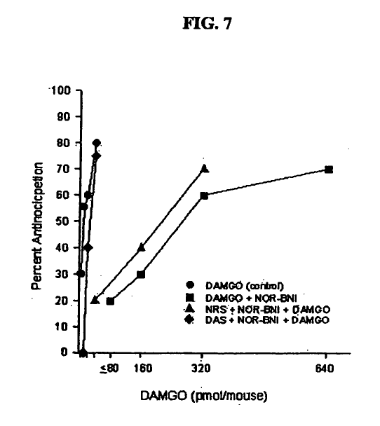

Table 1 shows the results of the assays. The potency ratios show that DAMGO was antagonized by nor-BNI (κ antagonist) and CTOP (μ antagonist), but not BNTX (μ₁ antagonist) or NTB (μ₂ antagonist). Inasmuch as DAMGO has been reported [Vanderah,T. W., Ossipov, M. H., Lai, J., Malan Jr., T. P. & Porreca, F. Pain, 92, 5-9 (2001)] to promote the release of spinal dynorphin-A, an assay in the presence of antiserum to dynorphin A was conducted to determine if the apparent antagonism by norBNI of DAMGO-induced antinoception was due to antagonism of dynorphin-A. With co-administration of norBNI with dynorphin-A antiserum (DAS), there was little if any antagonism of DAMGO antinociception (FIG. 7). This suggests the observed potent antagonism by nor-BNI in the absence of antiserum was due to DAMGO-promoted release of dynorphin-A whose acute antinociceptive effect was antagonized at κ receptors by norBNI. Thus, DAMGO does not interact with κ receptors, despite being antagonized by nor-BNI.

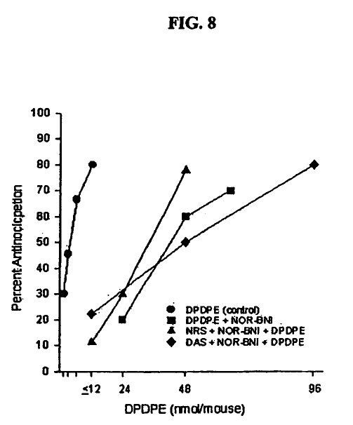

DPDPE was antagonized by nor-BNI (κ antagonist) and BNTX (δ₁ antagonist), but not NTB (δ₂ antagonist) or CTOP (μ antagonist). In contrast to the above results with the combination of DAMGO and nor-BNI, dynorphin-A antiserum (DAS) failed to substantially reduce the potent norBNI antagonism of DPDPE antinociception (FIG. 8). This indicates that dynorphin-A was not released in response to DPDPE agonism and that some other mechanism for the antagonism by norBNI is involved. Deltorphin II was antagonized only by NTB (δ₂ antagonist). U50488 was antagonized only by nor-BNI (κ antagonist).

TABLE 1
Antagonism of selective opioid agonist-induced antinociception
upon intrathecal (Hylden and Wilcox (1980) Eur. J. Pharmacol.
67: 313-316) administration in mice.

| | | ED₅₀ (nmol/mouse)^{c} | Potency Ratio^{d} |
|---|---|---|---|
| Agonist^{a} | Antagonist^{b} | (95% C.I.) | (95% C.I.) |
| DAMGO | nor-BNI | 0.283 (0.202-0.417) | 26.26 (16.30-46.90) |
| DPDPE | nor-BNI | 43.30 (36.40-51.59) | 12.44 (9.91-15.83) |
| Deltorphin II | nor-BNI | 3.07 (2.15-4.57) | 1.04 (0.62-1.77) |
| U50488 | nor-BNI | 106.14 (25.56-314.56) | 5.26 (0.24-16.04) |
| DAMGO | BNTX | 0.017 (0.012-0.027) | 1.50 (0.94-2.83) |
| DPDPE | BNTX | 41.10 (26.44-60.95) | 10.61 (6.08-18.51) |
| Deltorphin II | BNTX | 3.41 (2.12-6.12) | 0.99 (0.46-2.00) |
| U50488 | BNTX | 19.64 (15.93-25.18) | 0.94 (0.69-1.27) |
| DAMGO | NTB | 0.014 (0.008-0.027) | 1.20 (0.61-3.26) |
| DPDPE | NTB | 3.44 (2.69-4.45) | 0.98 (0.71-1.41) |
| Deltorphin II | NTB | 22.51 (16.34-30.41) | 9.07 (4.79-11.56) |
| U50488 | NTB | 17.80 (10.77-27.04) | 0.81 (0.33-1.50) |
| DAMGO | CTOP | 0.112 (0.070-0.157) | 9.89 (5.91-15.34) |
| DPDPE | CTOP | 5.11 (4.04-6.37)) | 1.43 (1.06-1.93) |
| Deltorphin II | CTOP | 5.65 (3.08-10.55) | 1.91 (0.78-4.47) |
| U50488 | CTOP | 19.44 (16.03-22.66) | 0.85 (0.69-1.03) |
| ^{a}Peak times and control ED₅₀'s (nmol/mouse) for the antinociceptive effect of the agonists (i.t.) were as follows: [D-Ala²,N-Me-Phe⁴,Gly-ol⁵]enkephalin (DAMGO), 20 min, 0.011 (0.007-0.015); [D-Pen^{2,5}]enkephalin (DPDPE), 10 min, 3.35 (3.05-3.66); [D-Ala², Glu⁴]deltorphin (Deltorphin II), | | | |
| #10 min, 2.91 (2.18-3.93); and 3,4-dichloro-N-methyl-N-[2-(1-pyrrolidiyl)cyclohexyl]benzeneacetamide (U50488), 12 min, 20.97 (18.18-24.71). | | | |

^{b}Peak times and the doses for the it. administered antagonists were as follows: norbinaltorphimine (nor-BNI), 2.5 nmol/mouse, 16 min; 7-benzylidenenaltrexone (BNTX), 25 pmoL/mouse, 10 min; Naltriben (NTB), 50 pmol/mouse, 10 min and D-Phe-Cys-Tyr-D-Trp-Orn-Thr-Phe-Thr-NH₂ (CTOP), 5.9 pmol/mouse, 20 min.
^{c,d}The parallel line assay of Finney (1964) analyzes quantal data and was used to estimate the ED₅₀ values and the 95% confidence intervals with the aid of a computer. Antagonism as expressed potency ratios (ED₅₀ with antagonist/control ED₅₀) was considered significant when the 95% confidence intervals of the rations was >1.0.

Conclusions

Since CTOP only antagonized DAMGO, and nor-BNI's antagonism of DAMGO was an artifact of DAMGO's stimulation of dynorphin-A release, we can conclude that nor-BNI does not interact with μ receptors. DAMGO is thought to be a selective μ agonist, and the results of this Example are consistent with that. U50488 is thought to be a κ agonist, and the results of this Example are consistent with that. DPDPE was antagonized by both a δ₁ antagonist and a κ0 antagonist, showing that the δ₁ receptor contains an accessible κ opioid receptor. In contrast, Deltorphin II was antagonized only by a δ₂ antagonist and not a κ antagonist. This shows the δ₂ receptor subtype does not contain an accessible κ opioid receptor.

These data demonstrate the presence of spinal δ-κ heteromers whose selectivity profile is consistent with that of the putative δ₁ receptor subtype.

All publications, patents, and patent documents are incorporated by reference herein, as though individually incorporated by reference (including PCT/US01/11339). The invention has been described with reference to various specific and preferred embodiments and techniques. However, it should be understood that many variations and modifications may be made while remaining within the spirit and scope of the invention.

1. A method for producing a selective analgesic effect outside the brain in a mammal, comprising administering to the mammal an effective analgesic dose of a compound that activates a delta-kappa opioid receptor in the mammal.

2-5. (Canceled)

6. The method of claim 1 wherein the compound binds to a delta-kappa opioid receptor at least 3-fold more strongly than it binds to a kappa receptor.

7. The method of claim 1 wherein the compound binds to a delta-kappa opioid receptor at least 5-fold more strongly than it binds to a kappa receptor.

8. The method of claim 1 wherein the compound binds to a delta-kappa opioid receptor at least 10-fold more strongly than it binds to a kappa receptor.

9. The method of claim 1 wherein the compound binds to a delta-kappa opioid receptor at least 3-fold more strongly than it binds to a delta receptor.

10. The method of claim 1 wherein the compound binds to a delta-kappa opioid receptor at least 5-fold more strongly than it binds to a delta receptor.

11. The method of claim 1 wherein the compound binds to a delta-kappa opioid receptor at least 10-fold more strongly than it binds to a delta receptor.

12. The method of claim 1 wherein the compound binds to a delta-kappa opioid receptor at least 3-fold more strongly than it binds to a mu receptor.

13. The method of claim 1 wherein the compound binds to a delta-kappa opioid receptor at least 5-fold more strongly than it binds to a mu receptor.

14. The method of claim 1 wherein the compound binds to a delta-kappa opioid receptor at least 10-fold more strongly than it binds to a mu receptor.

15-16. (Canceled)

17. The method of claim 1 wherein the compound is a compound of formula (I): 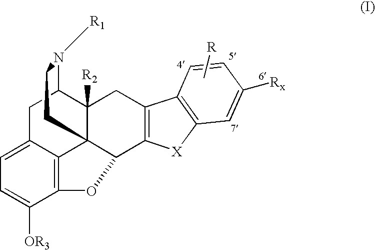
wherein:
R is hydrogen, halo, hydroxy, nitro, cyano, trifluoromethyl, trifluoromethoxy, (C₁-C₆)alkyl, (C₂-C₆)alkenyl, (C₂-C₆)alkynyl, (C₃-C₇)cycloalkyl, (C₅-C₇)cycloalkenyl, (C₃-C₆)cycloalkyl(C₁-C₆)alkyl, (C₅-C₇)cycloalkenyl(C₁-C₆)alkyl, aryl, heteroaryl, aryl(C₁-C₆)alkyl, heteroaryl(C₁-C₆)alkyl (C₁-C₆)alkoxy, (C₁-C₆)alkanoyloxy, NRₐR_{b} or SR_{c};
R₁ is (C₁-C₆)alkyl, (C₂-C₆)alkenyl, (C₂-C₆)alkynyl, (C₃-C₇)cycloalkyl, (C₅-C₇)cycloalkenyl, (C₃-C₆)cycloalkyl(C₁-C₆)alkyl, (C₅-C₇)cycloalkenyl(C₁-C₆)alkyl, aryl, heteroaryl, aryl(C₁-C₆)alkyl, or heteroaryl(C₁-C₆)alkyl;
R₂ is H, hydroxy, (C₁-C₆)alkoxy, (C₁-C₆)alkanoyloxy, NRₐR_{b} or SR_{c};
R₃ is H, aryl(C₁-C₆)alkyl, (C₁-C₆)alkyl, (C₁-C₆)alkanoyl, or (C₁-C₆)alkylC(═S);
Rₓ is a basic or positively charged group or an organic radical that comprises a basic or positively charged group;
X is O, S, CH₂, or NY;
Y is H, (C₁-C₆)alkyl, or aryl(C₁-C₆)alkyl; and
Rₐ—R_{c} are each independently H, (C₁-C₆)alkyl, (C₁-C₆)alkanoyl, phenyl, benzyl, phenethyl, or —C(═S)(C₁-C₆)alkyl;
or a pharmaceutically acceptable salt thereof.

18. The method of claim 17 wherein the basic or positively charged group is a quaternary amine or an amine salt.

19. The method of claim 17 wherein the compound is a 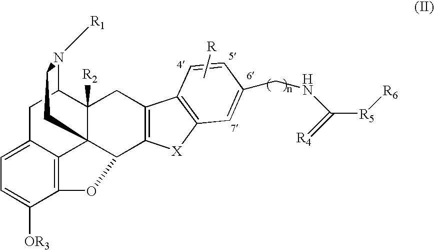
wherein:
R is hydrogen, halo, hydroxy, nitro, cyano, trifluoromethyl, trifluoromethoxy, (C₁-C₆)alkyl, (C₂-C₆)alkenyl, (C₂-C₆)alkynyl, (C₃-C₇)cycloalkyl, (C₅-C₇)cycloalkenyl, (C₃-C₆)cycloalkyl(C₁-C₆)alkyl, (C₅-C₇)cycloalkenyl(C₁-C₆)alkyl, aryl, heteroaryl, aryl(C₁-C₆)alkyl, or heteroaryl(C₁-C₆)alkyl (C₁-C₆)alkoxy, (C₁-C₆)alkanoyloxy, NRₐR_{b} or SR_{c};
R₁ is (C₁-C₆)alkyl, (C₂-C₆)alkenyl, (C₂-C₆)alkynyl, (C₃-C₇)cycloalkyl, (C₅-C₇)cycloalkenyl, (C₃-C₆)cycloalkyl(C₁-C₆)alkyl, (C₅-C₇)cycloalkenyl(C₁-C₆)alkyl, aryl, heteroaryl, aryl(C₁-C₆)alkyl, heteroaryl(C₁-C₆)alkyl;
R₂ is H, OH, (C₁-C₆)alkoxy, (C₁-C₆)alkanoyloxy, NRₐR_{b} or SR_{c};
R₃ is H, aryl(C₁-C₆)alkyl, (C₁-C₆)alkyl, (C₁-C₆)alkanoyl, or (C₁-C₆)alkylC(═S);
R₄ is ═O, ═S, or ═NR_{d};
R_{d} is H, CN, CONH₂, COCF₃, (C₁-C₆)alkanoyl, (C₁-C₆)alkyl, or (CH₂)ₚNRₑR_{f}; or R_{d} together with R₆ is —(CH₂)_{q}— and forms a ring;
p is 1, 2, 3, or 4;
R₅ is NRₘ;
R₆ is H, (C₁-C₆)alkyl, (C₃-C₇)cycloalkyl, aryl, heteroaryl, aryl(C₁-C₆)alkyl, heteroaryl(C₁-C₆)alkyl, NR_{g}Rₕ(C₁-C₆)alkyl, or C(═NRⱼ)NHRₖ; or when R₄ is ═NR_{d}, R₆ together with R_{d} is —(CH₂)_{q}— and forms a ring;
q is 2 or 3;
X is O, S, CH₂, or NY;
Y is H, (C₁-C₆)alkyl, or aryl(C₁-C₆)alkyl;
n is O, 1, 2, 3, or 4;
Rₐ—R_{c} and Rₑ—R_{f} are each independently H, (C₁-C₆)alkyl, (C₁-C₆)alkanoyl, phenyl, benzyl, phenethyl, or —C(═S)(C₁-C₆)alkyl;
R_{g} and Rₕ are each independently H, (C₁-C₆)alkyl, (C₁-C₆)alkanoyl, —C(═NH)NRₐR_{b}, or —C(═S)(C₁-C₆)alkyl, or R_{g} and Rₕ together with the nitrogen to which they are attached are pyrrolidino, piperidino or morpholino;
Rⱼ and Rₖ are each independently H, (C₁-C₆)alkyl, (C₂-C₆)alkenyl, (C₂-C₆)alkynyl, (C₃-C₇)cycloalkyl, (C₃-C₇)cycloalkyl(C₁-C₆)alkyl, (C₅-C₇)cycloalkenylalkyl, aryl, heteroaryl, aryl(C₁-C₆)alkyl, or heteroaryl(C₁-C₆)alkyl; and
Rₘ is hydrogen or (C₁-C₆)alkyl;
or a pharmaceutically acceptable salt thereof

20. The method of claim 17 wherein R is hydrogen or halo.

21. The method of claim 17 wherein R₁ is (C₂-C₆)alkenyl or (C₃-C₆)cycloalkyl(C₁-C₆)alkyl.

22. The method of claim 17 wherein R₁ is cyclopropylmethyl or allyl.

23. The method of claim 17 wherein R₂ is OH.

24. The method of claim 17 wherein R₃ is H.

25. The method of claim 19 wherein R₄ is ═NR_{d}.

26. The method of claim 19 wherein R₄ is ═NH or ═NCN.

27. The method of claim 19 wherein R₅ is NH.

28. The method of claim 19 wherein R₆ is H.

29. The method of claim 19 wherein R₆ is hydrogen, ethyl, n-butyl, 3-(dimethylamino)propyl, or 2-pyrrolidinoethyl.

30. The method of claim 19 wherein R₆ is C(═NRⱼ)NHRₖ.

31. The method of claim 19 wherein R_{d}, together with R₆, is —(CH₂)_{q}— and forms a ring.

32. The method of claim 19 wherein Rₘ is hydrogen.

33. The method of claim 19 wherein n is 0.

34. The method of claim 19 wherein n is 1.

35. The method of claim 19 wherein X is NH.

36. The method of claim 19 wherein R₁ is cyclopropylmethyl; R₂ is hydroxy; R₃ is H; R₄ is ═NH; R₅ is NH; and R₆ is H.

37. The method of claim 1 wherein the compound is
6′-guanidinyl-17-cyclopropylmethyl-6,7-didehydro-4,5-α-epoxy-3,14-dihydroxyindolo[2′,3′:6,7]morphinan;
6′-N-methylguanidinyl-17-cyclopropylmethyl-6,7-didehydro-4,5-α-epoxy-3,14-dihydroxyindolo[2′,3′:6,7]morphinan;
6′-N-ethylguanidinyl-17-cyclopropylmethyl-6,7-didehydro-4,5-α-epoxy-3,14-dihydroxyindolo[2 ′,3′:6,7]morphinan;
6′-N-propylguanidinyl-17-cyclopropylmethyl-6,7-didehydro-4,5-α-epoxy-3,14-dihydroxyindolo[2′,3′:6,7]morphinan;
6′-N-butylguanidinyl-17-cyclopropylmethyl-6,7-didehydro-4,5-α-epoxy-3,14-dihydroxyindolo[2′,3′:6,7]morphinan;
6′-N-pentylguanidinyl-17-cyclopropylmethyl-6,7-didehydro-4,5 -α-epoxy-3,14-dihydroxyindolo[2′,3′:6,7]morphinan;
6′-N-hexylguanidinyl-17-cyclopropylmethyl-6,7-didehydro-4,5-α-epoxy-3,14-dihydroxyindolo[2′,3′:6,7]morphinan;
6′-N′-cyano-N-[17-(cyclopropylmethyl)-6,7-didehydro-4,5α-epoxy-3,14-dihydroxyindolo[2′,3′:6,7]morphinian]-guanidine;
6′-N-cyano-N′-[3 -(dimethylaminopropyl)]-N″-[17-(cyclopropylmethyl)-6,7-didehydro-4,5α-epoxy-3,14-dihydroxyindolo[2′,3′:6,7]morphinian]-guanidine;
6′-N-cyano-N′-[2-(1-aminoethylpyrrolidine)]-N″-[17-(cyclopropylmethyl)-6,7-didehydro-4,5α-epoxy-3,14-dihydroxyindolo-[2′,3′:6,7]morphinian]-guanidine;
5′-Fluoro-6′-guanidino-17-(cyclopropylmethyl)-6,7-didehydro-4,5α-epoxy-3,14-hydroxyindolo-[2′,3′:6,7]morphinian; or
5′-Chloro-6′-guanidino-17-(cyclopropylmethyl)-6,7-didehydro-4,5α-epoxy-3,14-hydroxyindolo-[2′,3′:6,7]morphinian;
or a pharmaceutically acceptable salt thereof.

38. The method of claim 1 wherein the compound is 6′-guanidinyl-17-cyclopropylmethyl-6,7-didehydro-4,5-α-epoxy-3,14-hydroxyindolo-[2′,3′:6,7]morphinan; or a pharmaceutically acceptable salt thereof

39-43. (Cancel)

44. A compound of formula (I): 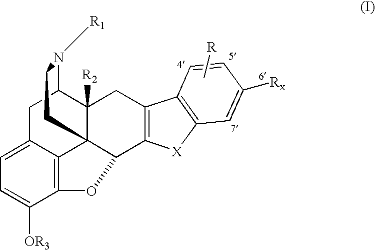
wherein:
R is hydrogen, halo, hydroxy, nitro, cyano, trifluoromethyl, trifluoromethoxy, (C₁-C₆)alkyl, (C₂-C₆)alkenyl, (C₂-C₆)alkynyl, (C₃-C₇)cycloalkyl, (C₅-C₇)cycloalkenyl, (C₃C₆)cycloalkyl(C₁-C₆)alkyl, (C₅-C₇)cycloalkenyl(C₁-C₆)alkyl, aryl, heteroaryl, aryl(C₁-C₆)alkyl, heteroaryl(C₁-C₆)alkyl (C₁-C₆)alkoxy, (C₁-C₆)alkanoyloxy, NRₐR_{b} or SR_{c};
R₁ is (C₁-C₆)alkyl, (C₂-C₆)alkenyl, (C₂-C₆)alkynyl, (C₃-C₇)cycloalkyl, (C₅-C₇)cycloalkenyl, (C₃-C₆)cycloalkyl(C₁-C₆)alkyl, (C₅-C₇)cycloalkenyl(C₁-C₆)alkyl, aryl, heteroaryl, aryl(C₁-C₆)alkyl, or heteroaryl(C₁-C₆)alkyl;
R₂ is H, hydroxy, (C₁-C₆)alkoxy, (C₁-C₆)alkanoyloxy, NRₐR_{b} or SR_{c};
R₃ is H, aryl(C₁-C₆)alkyl, (C₁-C₆)alkyl, (C₁-C₆)alkanoyl, or (C₁-C₆)alkylC(═S);
Rₓ is a basic or positively charged group or an organic radical that comprises a basic or positively charged group;
X is CH₂; and
Rₐ—R_{c} are each independently H, (C₁-C₆)alkyl, (C₁-C₆)alkanoyl, phenyl, benzyl, phenethyl, or —C(═S)(C₁-C₆)alkyl;
or a pharmaceutically acceptable salt thereof.

45. A compound of formula (I): 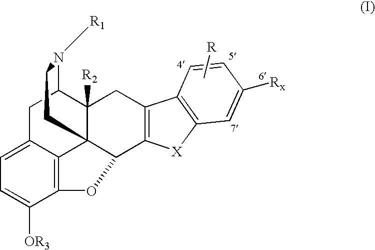
wherein:
R is halo, hydroxy, nitro, cyano, trifluoromethyl, trifluoromethoxy, (C₁-C₆)alkyl, (C₂-C₆)alkenyl, (C₂-C₆)alkynyl, (C₃-C₇)cycloalkyl, (C₅-C₇)cycloalkenyl, (C₃-C₆)cycloalkyl(C₁-C₆)alkyl, (C₅-C₇)cycloalkenyl(C₁-C₆)alkyl, aryl, heteroaryl, aryl(C₁-C₆)alkyl, heteroaryl(C₁-C₆)alkyl (C₁-C₆)alkoxy, (C₁-C₆)alkanoyloxy, NRₐR_{b} or S_{c};
R₁ is (C₁-C₆)alkyl, (C₂-C₆)alkenyl, (C₂-C₆)alkynyl, (C₃-C₇)cycloalkyl, (C₅-C₇)cycloalkenyl, (C₃-C₆)cycloalkyl(C₁-C₆)alkyl, (C₅-C₇)cycloalkenyl(C₁-C₆)alkyl, aryl, heteroaryl, aryl(C₁-C₆)alkyl, or heteroaryl(C₁-C₆)alkyl;
R₂ is H, hydroxy, (C₁-C₆)alkoxy, (C₁-C₆)alkanoyloxy, NRₐR_{b} or S_{c};
R₃ is H, aryl(C₁-C₆)alkyl, (C₁-C₆)alkyl, (C₁-C₆)alkanoyl, or (C₁-C₆)alkylC(═S);
Rₓ is a basic or positively charged group or an organic radical that comprises a basic or positively charged group;
X is O, S, CH₂, or NY;
Y is H, (C₁-C₆)alkyl, or aryl(C₁-C₆)alkyl; and
Rₐ—R_{c} are each independently H, (C₁-C₆)alkyl, (C₁-C₆)alkanoyl, phenyl, benzyl, phenethyl, or —C(═S)(C₁-C₆)alkyl;
or a pharmaceutically acceptable salt thereof.

46. The compound of claim 45 wherein R is halo, hydroxy, nitro, cyano, trifluoromethyl, trifluoromethoxy, (C₂-C₆)alkenyl, (C₂-C₆)alkynyl, (C₃-C₇)cycloalkyl, (C₅-C₇)cycloalkenyl, (C₃-C₆)cycloalkyl(C₁-C₆)alkyl, (C₅-C₇)cycloalkenyl(C₁-C₆)alkyl, aryl, heteroaryl, aryl(C₁-C₆)alkyl, heteroaryl(C₁-C₆)alkyl (C₁-C₆)alkoxy, (C₁-C₆)alkanoyloxy, NRₐR_{b} or SR_{c}.

47. The compound of claim 46 wherein R is halo, hydroxy, nitro, cyano, trifluoromethyl, trifluoromethoxy, (C₃-C₇)cycloalkyl, (C₅-C₇)cycloalkenyl, (C₃-C₆)cycloalkyl(C₁-C₆)alkyl, (C₅-C₇)cycloalkenyl(C₁-C₆)alkyl, aryl, heteroaryl, aryl(C₁-C₆)alkyl, heteroaryl(C₁-C₆)alkyl (C₁-C₆)alkoxy, (C₁-C₆)alkanoyloxy, NRₐR_{b} or S_{c};

48. The compound 5′-fluoro-6′-guanidino-17-(cyclopropylmethyl)-6,7-didehydro-4,5α-epoxy-3,14-hydroxyindolo-[2′,3′:6,7]morphinian; or 5′-fhloro-6′-guanidino-17-(cyclopropylmethyl)-6,7-didehydro-4,5α-epoxy-3,14-hydroxyindolo-[2′,3′:6,7]morphinian; or a pharmaceutically acceptable salt thereof.

49. A compound of formula (I): 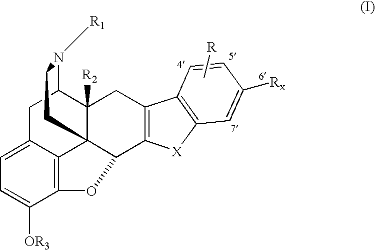
wherein:
R is hydrogen, halo, hydroxy, nitro, cyano, trifluoromethyl, trifluoromethoxy, (C₁-C₆)alkyl, (C₂-C₆)alkenyl, (C₂-C₆)alkynyl, (C₃-C₇)cycloalkyl, (C₅-C₇)cycloalkenyl, (C₃-C₆)cycloalkyl (C₁-C₆)alkyl, (C₅-C₇)cycloalkenyl(C₁-C₆)alkyl, aryl, heteroaryl, aryl(C₁-C₆)alkyl, heteroaryl(C₁-C₆)alkyl (C₁-C₆)alkoxy, (C₁-C₆)alkanoyloxy, NRₐR_{b} or SR_{c};
R₁ is (C₁-C₆)alkyl, (C₂-C₆)alkenyl, (C₂-C₆)alkynyl, (C₃-C₇)cycloalkyl, (C₅-C₇)cycloalkenyl, (C₃-C₆)cycloalkyl(C₁-C₆)alkyl, (C₅-C₇)cycloalkenyl(C₁-C₆)alkyl, aryl, heteroaryl, aryl(C₁ -C₆)alkyl, or heteroaryl(C₁-C₆)alkyl;
R₂ is H, hydroxy, (C₁-C₆)alkoxy, (C₁-C₆)alkanoyloxy, NRₐR_{b} or SR_{c};
R₃ is H, aryl(C₁-C₆)alkyl, (C₁-C₆)alkyl, (C₁-C₆)alkanoyl, or (C₁-C₆)alkylC(═S);
Rₓ is a basic or positively charged group or an organic radical that comprises a basic or positively charged group;
X is O, S, CH₂, or NY;
Y is H, (C₁-C₆)alkyl, or aryl(C₁-C₆)alkyl; and
Rₐ—R_{c} are each independently H, (C₁-C₆)alkyl, (C₁-C₆)alkanoyl, phenyl, benzyl, phenethyl, or —C(═S)(C₁-C₆)alkyl;
or a pharmaceutically acceptable salt thereof.
wherein Rₓ is not —(CH₂)ₙ—NH—C(═R₄)—R₅—R₆,
wherein
n is 0, 1, 2, 3, 4;
R₄ is ═O, ═S, or ═NR_{d};
R_{d} is H, CN, CONH₂, COCF₃, (C₁-C₆)alkanoyl, (C₁-C₆)alkyl, or (CH₂)ₚNRₑR_{f}, or
R_{d} together with R₆ is —(CH₂)_{q}— and forms a ring;
p is 1, 2, 3, or 4;
R₅ is NRₘ;
R₆ is H, (C₁-C₆)alkyl, (C₃-C₇)cycloalkyl, aryl, heteroaryl, aryl(C₁-C₆)alkyl, heteroaryl(C₁-C₆)alkyl, NR_{g}Rₕ(C₁-C₆)alkyl, or C(═NRⱼ)NHRₖ; or when R₄ is ═NR_{d}, R₆ together with R_{d} is —(CH₂)_{q}— and forms a ring;
q is 2 or 3;
Rₑ—R_{f} are each independently H, (C₁-C₆)alkyl, (C₁-C₆)alkanoyl, phenyl, benzyl, phenethyl, or —C(═S)(C₁-C₆)alkyl;
R_{g} and Rₕ are each independently H, (C₁-C₆)alkyl, (C₁-C₆)alkanoyl, —C(═NH)NRₐR_{b}, or —C(═S)(C₁-C₆)alkyl, or R_{g} and Rₕ together with the nitrogen to which they are attached are pyrrolidino, piperidino or morpholino;
Rⱼ and Rₖ are each independently H, (C₁-C₆)alkyl, (C₂-C₆)alkenyl, (C₂-C₆)alkynyl, (C₃-C₇)cycloalkyl, (C₃-C₇)cycloalkyl(C₁-C₆)alkyl, (C₅-C₇)cycloalkenylalkyl, aryl, heteroaryl, aryl(C₁-C₆)alkyl, or heteroaryl(C₁-C₆)alkyl; and
Rₘ is hydrogen or (C₁-C₆)alkyl.

50. A pharmaceutical composition comprising a compound as described in any one of claims 44-49, and a pharmaceutically acceptable carrier.

51. (Canceled)

52. A method for producing an analgesic effect in a mammal via selective agonism of opioid receptors in the spinal cord of the mammal, comprising administering to the mammal an effective analgesic dose of a compound that selectively activates delta-kappa opioid receptors.

53. A method for producing spinal analgesia in a mammal comprising administering to the mammal an effective analgesic dose of a compound that activates delta-kappa opioid receptors.

54. A method to produce selective agonism of opioid receptors outside the brain in a mammal comprising administering to the mammal an effective dose of a compound that activates delta-kappa opioid receptors.

55. A method to produce spinal analgesia in a mammal comprising administering to the mammal an effective dose of a compound that selectively activates opioid receptors in the spine.

56. (Canceled)
